(19) United States
(12) Patent Application Publication
(10) Pub. No.: US 2025/0391024 A1
(43) Pub. Date: Dec. 25, 2025

(54) SOFT TISSUE MODELLING

(71) Applicant: FORMUS IP LIMITED, Grafton (NZ)

(72) Inventors: Ju ZHANG, Auckland (NZ); Thor Franciscus BESIER, Auckland (NZ)

(21) Appl. No.: 19/317,642

(22) Filed: Sep. 3, 2025

Related U.S. Application Data
(63) Continuation of application No. PCT/NZ2024/050027, filed on Mar. 4, 2024.

(30) Foreign Application Priority Data
Mar. 3, 2023 (NZ) 797763

(51) Int. Cl. G06T 7/00; G06T 7/11

(52) U.S. Cl. CPC G06T 7/0012; G06T 7/11; G06T 2207/20056; G06T 2207/20081; G06T 2207/30008

(57) ABSTRACT

A method implemented on an electronic computing device for estimating a force potential of a muscle or muscle group is described. The method comprising: receiving an image of a patient, the image depicting at least a portion of a muscle or muscle group; segmenting the image of the patient to produce a segmented patient image, the segmented patient image comprising at least one segmented muscle or segmented muscle group; and estimating a muscle force potential for the segmented muscle or segmented muscle group, wherein the estimated muscle force potential is based at least partially on the segmented patient image.

### CROSS-REFERENCE TO RELATED APPLICATIONS

This is a bypass continuation of International PCT Application No. PCT/NZ2024/050027, filed on Mar. 4, 2024, which claims priority to New Zealand Patent Application No. PCT/NZ2024/050027, filed on Mar. 3, 2023, which are incorporated by reference herein in their entirety.

### FIELD

This invention relates to estimating the characteristics of soft-tissue structures.

### SUMMARY

According to one example there is provided a method implemented on an electronic computing device for estimating a force potential of a muscle or muscle group, the method comprising: receiving an image of a patient, the image depicting at least a portion of a muscle or muscle group, segmenting the image of the patient to produce a segmented patient image, the segmented patient image comprising at least one segmented muscle or segmented muscle group, and estimating a muscle force potential for the segmented muscle or segmented muscle group; wherein the estimated muscle force potential is based at least partially on the segmented patient image.

Examples may be implemented according to any one of dependent claims 2 to 14.

According to another example there is provided a method implemented on an electronic computing device for estimating a passive tension of a soft-tissue structure, the method comprising: receiving an image of a patient, the image depicting at least a portion of a soft tissue structure, segmenting the image of the patient to produce a segmented patient image, the segmented patient image comprising at least one segmented soft-tissue structure, and estimating a passive tension for the segmented soft-tissue structure; wherein the estimated passive tension is based at least partially on the segmented patient image.

Examples may be implemented according to any one of dependent claims 16 to 27.

According to another example there is provided a method implemented on an electronic computing device for developing a pre-operative plan for a patient, the method comprising: receiving a pre-operative image of the patient, the image depicting at least a portion of a soft-tissue structure, segmenting the pre-operative image of the patient to produce a segmented pre-operative patient image, the segmented pre-operative patient image comprising at least one segmented soft-tissue structure, estimating a pre-operative passive tension for the segmented soft-tissue structure, wherein the estimated pre-operative passive tension is based at least partially on the segmented pre-operative patient image, and determining at least one target surgical parameter based at least partially on the estimated pre-operative passive tension for the segmented soft-tissue structure.

Examples may be implemented according to any one of dependent claims 29 to 44.

According to another example there is provided a method implemented on an electronic computing device for estimating a post-operative passive tension of a soft-tissue structure, the method comprising: receiving a pre-operative surgical plan for a patient, the pre-operative surgical plan comprising a target surgical parameter for soft-tissue structure, receiving a pre-operative image of the patient, the image depicting the soft-tissue structure, segmenting the pre-operative image of the patient to produce a segmented pre-operative patient image, the segmented pre-operative patient image comprising at least one segmented soft-tissue structure, estimating a pre-operative passive tension for the segmented soft-tissue structure, wherein the estimated pre-operative passive tension is based at least partially on the segmented pre-operative patient image, and estimating a post-operative passive tension for the segmented soft-tissue structure, wherein the estimated post-operative passive tension is based at least partially on the estimated pre-operative passive tension and the target surgical parameter.

Examples may be implemented according to any one of dependent claims 46 to 59.

According to another example there is provided a method implemented on an electronic computing device for estimating a muscle quality of a muscle or muscle group, the method comprising: receiving an image of a patient, the image at least partially depicting a muscle or muscle group, segmenting the image of the patient to produce a segmented patient image, the segmented patient image comprising at least one segmented muscle or segmented muscle group, and estimating a muscle quality for the segmented muscle or segmented muscle group; wherein the estimated muscle quality is based at least partially on the segmented patient image.

Examples may be implemented according to any one of dependent claims 61 to 77.

According to another example there is provided a method implemented on an electronic computing device for estimating pathing of a muscle or muscle group, the method comprising: receiving an image of a patient, the image at least partially depicting at least a portion of a muscle or muscle group, segmenting the image of the patient to produce a segmented patient image, the segmented patient image comprising at least one segmented muscle or segmented muscle group, and estimating a pathing for the segmented muscle or segmented muscle group; wherein the estimated pathing is based at least partially on the segmented patient image.

Examples may be implemented according to any one of dependent claims 79 to 89.

According to another example there is provided a method implemented on an electronic computing device for training a machine learning model, the method comprising: receiving a plurality of 3D CT images, wherein each 3D CT image at least partially depicts at least one soft-tissue structure; producing, for each 3D CT image, at least one synthetic X-ray image; producing a training dataset by associating each 3D CT image with its associated at least one synthetic X-ray image; and training a machine learning model using the training dataset.

Examples may be implemented according to any one of dependent claims 91 to 96.

According to another example there is provided a method implemented on an electronic computing device for extracting soft-tissue data using a machine learning model, the method comprising: receiving at least one 2D X-ray image of a patient; producing, using a machine learning model, at least one 3D CT image based at least partially on the 2D X-ray image, wherein the at least one 3D CT image depicts at least a portion of a soft-tissue structure; segmenting the 3D CT image to produce a segmented patient image, the segmented patient image comprising at least one segmented soft-tissue structure; and estimating a soft-tissue trait based at least partially on the segmented patient image.

Examples may be implemented according to any one of dependent claims 98 to 101.

According to another example there is provided a method implemented on an electronic computing device for training a machine learning model, the method comprising: receiving a plurality of 3D CT images, wherein each 3D CT image at least partially depicts at least one soft-tissue structure; producing, for each 3D CT image, at least one synthetic X-ray image; producing, for each 3D CT image, at least one 3D soft-tissue mask volume; producing a training dataset by associating each at least one 3D soft-tissue mask volume with the at least one synthetic X-ray image originating from the same 3D CT image; and training a machine learning model using the training dataset.

Examples may be implemented according to any one of dependent claims 103 to 108.

According to another example there is provided a method implemented on an electronic computing device for extracting soft-tissue data using a machine learning model, the method comprising: receiving at least one 2D X-ray image of a patient; producing, using a machine learning model, at least one 3D soft-tissue mask volume based at least partially on the 2D X-ray image; and estimating a soft-tissue trait based at least partially on the at least one 3D soft-tissue mask volume.

Examples may be implemented according to any one of dependent claims 110 to 113.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings, which are incorporated in and constitute part of the specification, illustrate embodiments of the invention and, together with the general description of the invention given above, and the detailed description of embodiments given below, serve to explain the principles of the invention, in which:

FIG. 1A illustrates an example networked data processing environment.

FIG. 1B illustrates an example method implemented on an electronic computing device for estimating a force potential of a muscle or muscle group.

FIG. 2 illustrates an example method implemented on an electronic computing device for estimating pathing of a muscle or muscle group.

FIG. 3 illustrates an example method implemented on an electronic computing device for estimating a muscle quality for a muscle or muscle group.

FIG. 4 illustrates an example method implemented on an electronic computing device for estimating a passive tension of a soft-tissue structure.

FIG. 5 illustrates an example method implemented on an electronic computing device for estimating a post-operative passive tension of a soft-tissue structure.

FIG. 6 illustrates an example method implemented on an electronic computing device for determining a target surgical parameter.

FIG. 7 illustrates an example system.

FIG. 8 illustrates an example electronic computing device.

FIG. 9A illustrates an example method for training a machine learning model.

FIG. 9B illustrates an example method of extracting soft-tissue data using a machine learning model.

FIG. 9C illustrates a further example method for training a machine learning model.

FIG. 9D illustrates a further example method of extracting soft-tissue data using a machine learning model.

### DETAILED DESCRIPTION

The methods and systems disclosed herein generally relate to estimating and determining the properties of soft-tissue structures of patients from pre-operative and post-operative patient images. Understanding these soft tissue properties can be useful for pre-operatively planning surgeries, determining effective post-operative rehabilitation, and generally advising surgeons.

The methods disclosed herein can be implemented by instructions on a computing device, such as an electronic computing device. These can constitute a computer program that is embodied in various media, including tangible or non-tangible media, transitory or non-transitory media, and can run on any suitable device.

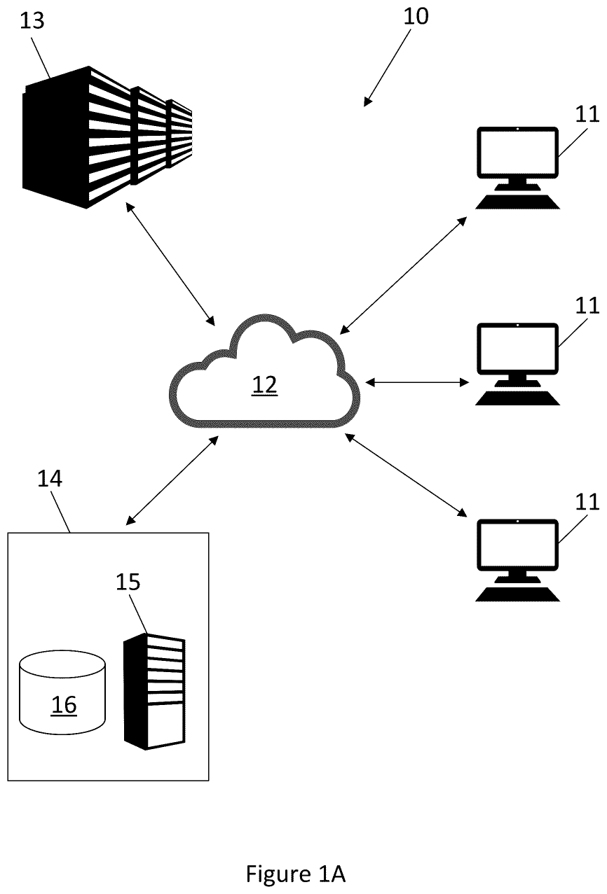

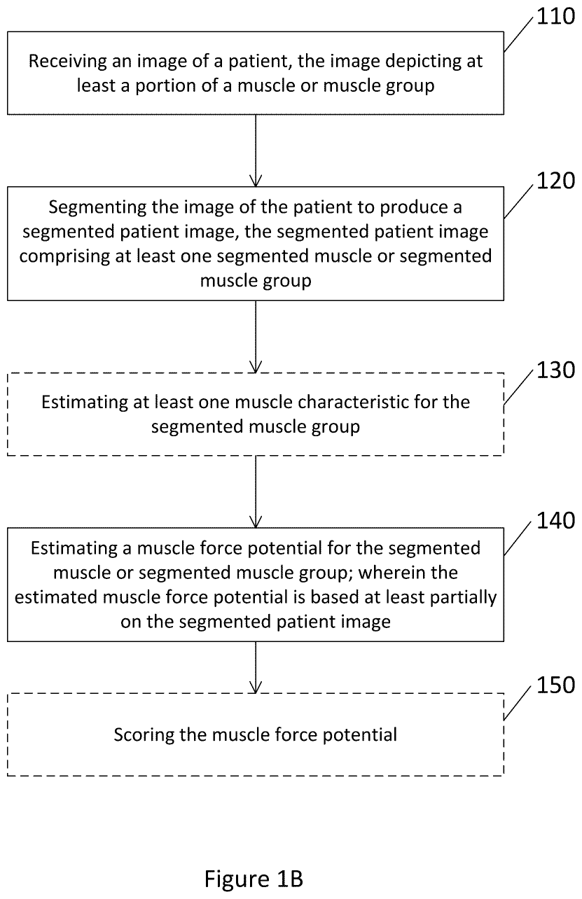

FIG. 1A depicts an example of networked data processing environment in which the methods disclosed herein can be implemented. The data processing environment 10 comprises a plurality of electronic computing devices 11 that can communicate over at least one network 12 via a wireless or wired connection. The plurality of electronic computing devices can be made available for users (e.g. surgeons). The at least one network 12 can be, for example, a wide area network (WAN) or a local area network (LAN). The data processing environment 10 further comprises at least one server 13 that is also configured to communicate over at least one network 12. The at least one server 13 can receive and handle requests for data processing and information management sent by electronic computing devices 11 via the at least one network 12. The data processing environment 10 can further comprise a third-party information source 14 configured to communicate over the at least one network 12. The third-party information source 14 can comprise data processing services 15 and can include a data store 16 for storing data.

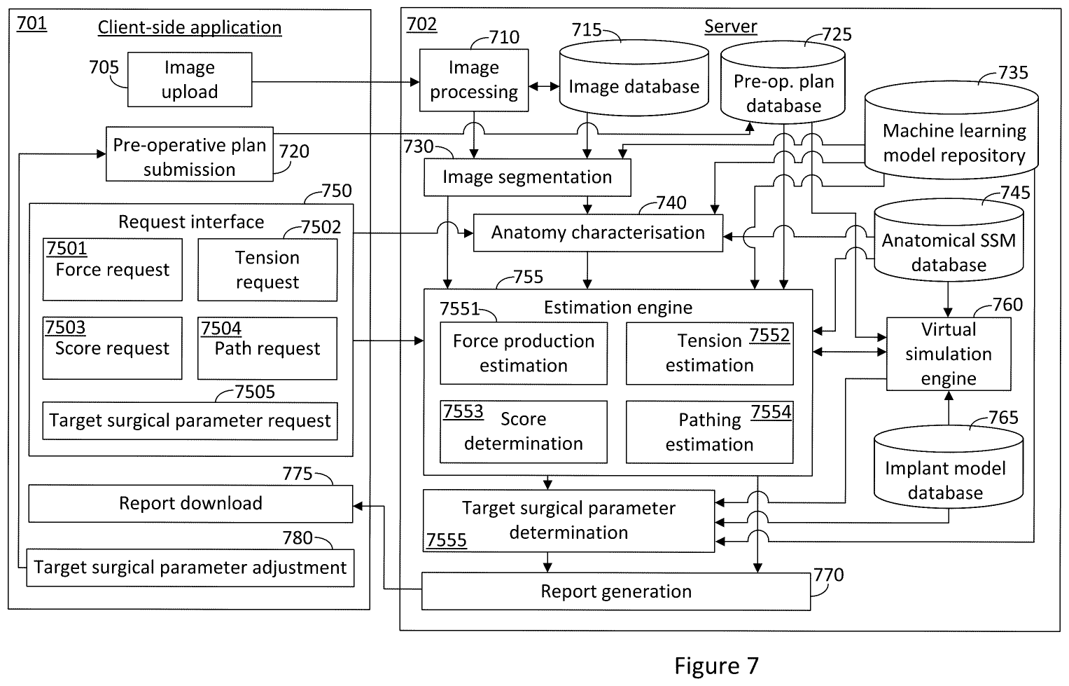

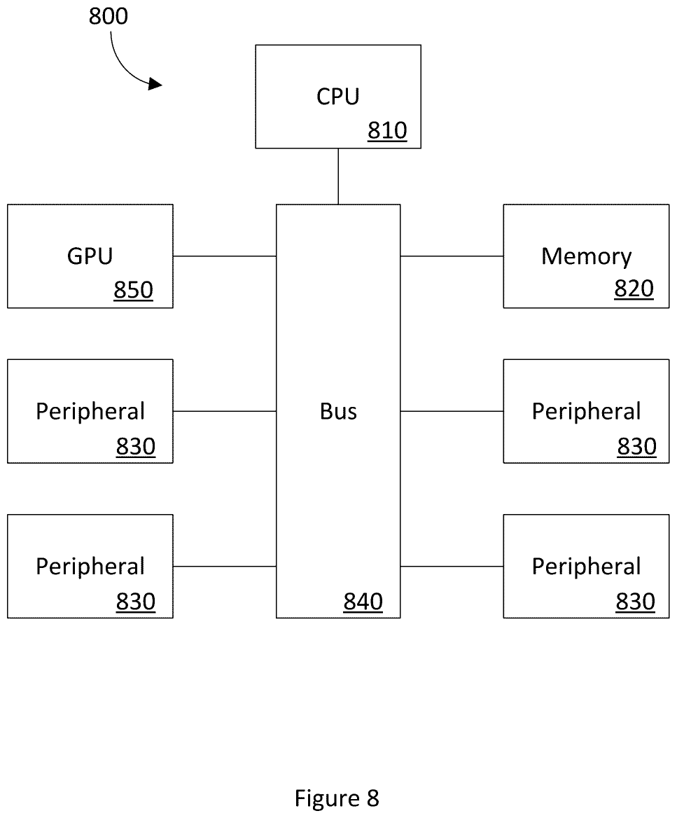

An example system configured to implement the methods disclosed herein is depicted in FIG. 7 and is described in more detail herein. Furthermore, an example architecture for an electronic computing device 11 and/or server 12 is depicted in FIG. 8 and is described in more detail herein.

Assessing Muscles from Images

FIG. 1B depicts one example method implemented on an electronic computing device for estimating a muscle force potential of a muscle or muscle group.

An image of the patient is received at 110. The image depicts at least a portion of a muscle or muscle group. The image is then segmented at 120 to produce a segmented patient image. The segmented patient image comprises at least one segmented muscle or muscle group. In some examples, at least one muscle characteristic can be estimated at 130 for the segmented muscle or segmented muscle group, based on the segmented patient image. A muscle force potential is then estimated at 140 for the segmented muscle or segmented muscle group. The estimated muscle force potential is based at least partially on the segmented patient image. If a muscle characteristic is estimated at 130, then the muscle force potential estimated at 140 can also be based at least partially on the muscle characteristic estimated at 130. The muscle force potential estimated at 140 can optionally be scored at 150.

The image received at 110 may be taken pre-operatively or post-operatively, and may be 2D or greater than 2D (e.g. 2.5D or 3D). In some examples, the image can be a computerised tomographic (CT) image or scan, a magnetic resonance image (MRI), ultrasound, body surface scan, or other suitable modality. In still further examples, the image can be a radiographic image (e.g. an X-ray). Although X-rays may not be conventionally used to image soft tissues due to insufficient tissue contrast, in some examples, a machine learning model can be trained to identify soft tissue structures within X-ray images, as described in more detail herein.

In still further examples, a combination of different imaging modalities can be used, or multiple images can be taken using a single modality, to construct the image received at 110. For example, multiple X-ray images can be taken from different angles and/or from different orientations, including neutral orientations. These X-ray images can be combined using stereophotogrammetry to produce three-dimensional image data. Three-dimensional data can also be determined from other imaging techniques using stereophotogrammetry or other applicable techniques.

In some examples, the segmented patient image can be produced at 120 using a machine learning model. For example, a convolutional neural network (CNN) can be trained to identify bulk muscle volumes within the image received at 110 and to segment the bulk muscle volumes into individual muscles and/or muscle groups. Suitable training data can include images that have been manually segmented and labelled. Alternatively or additionally, the machine learning model can be trained to segment the image of the patient into different tissues such as bone, muscle, sinews/connective tissues, and other tissues.

In other examples, the segmented patient image can be produced at 120, alternatively or additionally using one or more algorithms that are not based on machine learning models. For example, algorithms that use graph cuts (such as e.g. GrabCut) can be used to separate bulk muscle volume depicted in the image received at 110 from other tissues such as bone, fat, skin, and internal organs. A distribution of voxel intensities for each tissue can be calculated from a training set of manually segmented images. Machine learning models can also be used in conjunction with other algorithms. For example, after the image received at 110 is segmented using e.g. GrabCut, the segmented image can be further segmented using e.g. a CNN to produce a segmented patient image.

The segmented patient image produced at 120 comprises at least one segmented muscle or segmented muscle group. In some examples, only a portion of a segmented muscle may be visible (if, for example, the muscle or muscle group was only partially imaged in the image received at 110). In these examples, the remainder of the segmented muscle can be inferred or modelled using statistical shape modelling of the corresponding muscle/muscle group.

Examples of muscle characteristics that can optionally be estimated at 130 can include muscle volume, pennation angle, and/or muscle shape. In examples where a muscle characteristic is estimated at 130 for the segmented muscle or segmented muscle group, the muscle characteristic can be estimated using a machine learning model. For example, a machine learning model such as a deep neural network (DNN) or CNN can be trained to output one or more estimated muscle characteristics given a segmented muscle or segmented muscle group as an input. In some examples, the machine learning model can be trained via supervised learning by using a dataset of segmented images depicting segmented muscles/muscle groups labelled with qualitative or quantitative muscle characteristics. The machine learning model can then correlate aspects of the segmented patient image produced at 120, such as pixel or voxel count, position, intensity/colour, etc, with at least one muscle characteristic. For instance, a machine learning model can be trained to estimate muscle volume and/or shape from a 2D segmented patient image based on, e.g., the number, intensity, and location of pixels within the segmented muscle.

In other examples, a muscle characteristic can be estimated at 130 without using a machine learning model, or by using one or more algorithms in conjunction with a machine learning model. For instance, in some examples, a pennation angle can be estimated at 130 using a computational flow simulation. The initial conditions of the computational flow simulation can be inferred with the help of a machine learning model or can be algorithmically derived directly from the segmented patient image. Alternatively, a pennation angle can be estimated at 130 using a fast Fourier transform (FFT) on the segmented muscle region depicted within the segmented patient image. The pennation angle can then be determined from the direction of the highest spatial frequency within the transformed image.

In still further examples, muscle characteristics can be estimated at 130 using statistical shape models (SSMs). For example, the SSM can include a mean 3D shape of an anatomical component (e.g. a given bone, muscle, or other soft tissue) measured across a relevant population, alongside a description of the modes of variation of that mean shape observed across the population. The modes of variation can be determined using standard approaches such as principal component analysis. The shape of the segmented muscle or segmented muscle group can then be estimated by fitting the canonical representation of the segmented muscle or muscle group described by the SSM to landmarks derived from the segmented patient image by morphing the mean shape of the anatomical component according to the modes of variation described by the SSM, with each mode of variation weighted by a different score. Statistical shape modelling can also or alternatively be used to estimate other muscle characteristics at 130, such as e.g. muscle volume.

In some examples, the muscle force potential can be estimated at 140 using a machine learning model, such as a CNN, which receives the segmented patient image as an input. For example, a CNN can be trained via supervised learning using a dataset of segmented images depicting muscles or muscle groups that are labelled with muscle force potentials. In some examples, the muscle force potentials used in the training data can be derived from empirical measurements. For example, the training data can be produced by imaging a muscle for a cohort of people and recording the force potential for that muscle for each person within the cohort. The patient images can then be labelled with the recorded force potential for training data. In other examples, the muscle force potential used in the training data can be derived from e.g. dynamic simulations of virtual models of muscles. For example, for each training image depicting a muscle, a virtual model of the muscle can be constructed from the segmented image and simulated to estimate a force potential for the muscle. The estimated force potential can then be used as a label for the training data. This can allow the machine learning model to estimate the force potential of a segmented muscle or segmented muscle group within the segmented patient image, without needing to construct a virtual model of the muscle. Other examples where a machine learning model is used to estimate the muscle force potential at 140 can use other training data and/or other modalities of machine learning.

In examples where a muscle characteristic is estimated at 130, the muscle force potential can be estimated at 140 using a machine learning model that also uses muscle characteristics as inputs. For example, as the force potential of a muscle or muscle group can be related to the shape, volume, and/or pennation angle of the muscle or muscle group, a machine learning model can be trained to estimate the force potential of the muscle/muscle group taking these characteristics as additional inputs. For instance, the machine learning model may take pixel/voxel data from the segmented patient image as an input, in addition to one or more muscle characteristics estimated at 130.

In still further examples, the muscle force potential can be estimated at 140 without the use of a machine learning model. For example, in cases where muscle characteristics are estimated at 130 from the segmented patient image, the muscle force potential can be estimated at 140 using a mathematical equation or relationship describing the muscle/muscle group (e.g. using a Hill-type representation of the muscle). In other examples, the muscle force potential can be estimated at 140 using statistical modelling given the muscle characteristics estimated at 130. For example, if the patient's muscle volume, muscle shape, and/or pennation angle estimated at 130 can be expressed relative to the mean of a representative population, then a statistical muscle force potential can be estimated at 140 based on statistical relationships between the muscle force potential and muscle characteristics.

The optional scoring of the muscle force potential at 150 can also be determined using statistical modelling. For example, if the muscle force potential 140 can be expressed relative to the mean of a representative population, then the muscle force potential 140 can be assigned a Z-score. The score can be expressed quantitatively (e.g. using a Z-score) or qualitatively, such as e.g. ‘strong’, ‘average’, or ‘weak’. For instance, if the muscle has a Z-score of between e.g. Z=+1 and Z=+2, then the muscle may be classified as ‘strong’. In still further examples, the score attributed to the muscle force potential can be referenced against a context-dependent standard, such as a biomechanical task involving the use of the muscle, or in the context of a proposed surgery. For example, the force potential of a given muscle can be scored in the context of post-operative functionality in biomechanical tasks. If the force potential is estimated to be insufficient for one or more tasks, the score at 150 may be ‘weak’ and rehabilitation exercises can be recommended.

In further examples, an electronic computing device can be used to additionally or alternatively estimate a pathing of a muscle based on an image of a patient. The estimated pathing of muscle can be used, for example, to accurately describe the line of action and moment arm of the muscle. This can be useful for constructing virtual models of the patient anatomy or for estimating functionality of muscle.

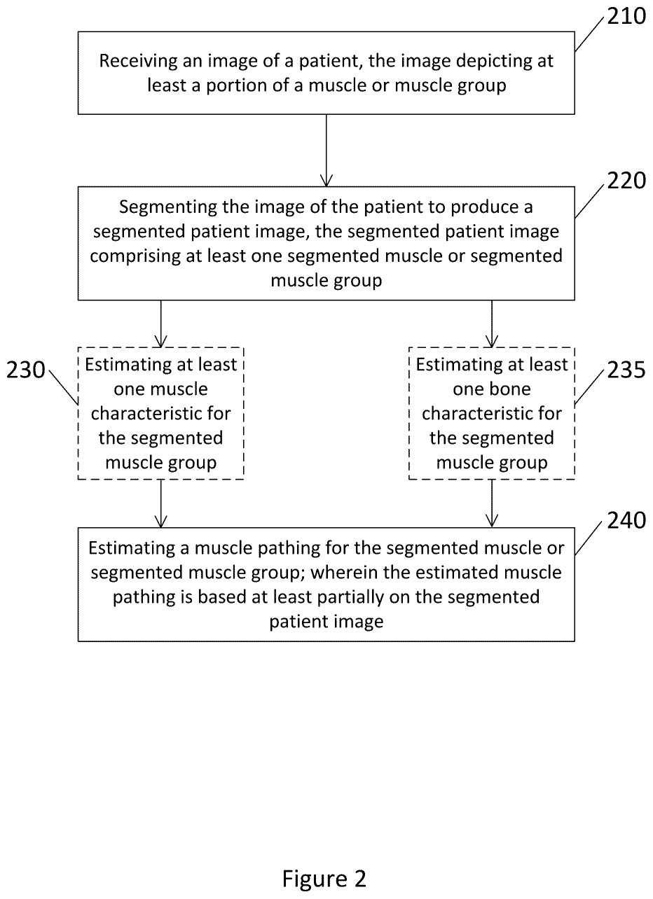

To this end, FIG. 2 depicts an example method implemented on an electronic computing device for estimating a pathing for a muscle or muscle group. An image of a patient is received at 210. The image depicts at least a portion of a muscle or muscle group and can also depict other anatomical features, such as adjacent or nearby muscles or soft tissue structures, and/or bony structures. The image is then segmented at 220 to produce a segmented patient image. The segmented patient image comprises at least one segmented muscle or segmented muscle group. In some examples, at least one muscle characteristic can be estimated at 230 for the segmented muscle or segmented muscle group, based on the segmented patient image. In some further examples, at least one bone characteristic can be estimated at 235 for at least one bony structure associated with the segmented muscle or segmented muscle group. A pathing is then estimated at 240 for the segmented muscle or segmented muscle group. The estimated pathing is based at least partially on the segmented patient image. If a muscle characteristic and/or bone characteristic is estimated at 230 or 235 respectively, then the pathing estimated at 240 can also be based at least partially on the muscle characteristic estimated at 230 and/or bone characteristic estimated at 235.

The image of the patient that is received at 210 can substantially be an image as has been described with respect to 110 of FIG. 1B. In addition to at least partially depicting at least a portion of the muscle or muscle group, the image received at 210 can also depict at least a portion of at least one bony structure associated with the at least one muscle or muscle group. For example, the image received at 210 can depict at least a portion of a bony structure to which the at least one muscle or muscle group attaches. The image received at 210 can further depict one or more points at which the at least one muscle or muscle group attaches to the bony structure.

The image can then be segmented at 220 in substantially the same way as described as 120 with respect to FIG. 1B. If the image received at 210 does depict a portion of a bony structure, then the segmentation process may segment and preserve the bony structure within the image so that the segmented patient image produced at 220 comprises at least one segmented bony structure. In some examples, the segmentation process at 220 can also segment interfaces of the muscle and bones.

In some examples, the image received at 210 and/or segmented at 220 may only depict or comprise a portion of a bony structure and/or muscle. In these instances, statistical shape models can be used to infer or model the remainders of the segmented muscles and/or segmented bone structures. For instance, the image received at 210 and/or segmented at 220 may depict e.g. only a portion of a tibial shaft without depicting e.g. the tibial plateau. In this case, an SSM can be fit to the visible portion of the patient's tibial shaft. The remainder of the tibia which is not depicted in the image received at 210 and/or segmented at 220—including the tibial plateau—can then be inferred based on the morphed mesh of the SSM to the representation of the tibial shaft. This can be useful if determining muscle characteristics at 230 and/or bone characteristic at 235 as described below.

Examples of muscle characteristics that can be optionally estimated at 230 can include muscle bulk shape and/or any other muscles (or soft-tissue structures) that the muscle wraps around. As with respect to 130 of FIG. 1B, the muscle characteristics can be estimated using a trained machine learning model in some examples. For example, a machine learning model can be trained to estimate bulk muscle shape from segmented patient images using pixel data. Additionally or alternatively, a muscle characteristic can be estimated using a statistical shape modelling approach as has been described with respect to 130 of FIG. 1B.

Similarly, bone characteristics that can be optionally estimated at 235 can include a shape of a bone, a location of an origin/insertion point, a shape of a bone at an origin/insertion point, and/or any other bony structures that the muscle wraps around. In some examples, the shape of the bone can be estimated using an SSM and/or using a trained machine learning model. Similarly, the location of an origin or insertion point and/or the shape of the bone at an origin/insertion point can be inferred using an SSM or by using a machine learning model.

In examples where only a portion of a bony structure is present in the image received at 210 and/or segmented at 220, the image may not depict an origin or insertion point, or may not depict the required portion of the bony structure necessary to determine a bone characteristic at 235. For instance, to continue the example given above, the tibial plateau (and other portions of the tibia) can be inferred by fitting an SSM model to the visible portion of a tibial shaft shown in a segmented image. The location of an origin point for a tibialis anterior muscle, and/or the shape of the bone (e.g. lateral condyle) at the origin point, can then be determined from the remainder of the tibia inferred via the SSM to estimate a bone characteristic at 235. Other muscles and/or bony structures can be inferred in the same way to estimate bone characteristics at 235.

In some examples, the SSM can include predefined origin and insertion points located on the anatomical mesh representative of the shape models. In these cases, the origin and/or insertion points can be defined once the SSM has been fit to the patient's anatomy depicted in the image segmented at 220.

In some further examples, origin and insertion points can be determined by using a machine learning model trained to identify origin/insertion points from segmented images. In some examples, the machine learning model can be a specialised model trained to specifically identify origin/insertion points for the particular bone in question, or bones of a similar class. In other examples, the machine learning model can be a general machine learning model trained to identify origin/insertion points from segmented images, without reference to any one specific kind of bone.

Muscle pathing is then estimated at 240. In some examples, the muscle pathing is based on the origin and insertion points for the segmented muscle or segmented muscle group. One or more centrelines through the muscle, running from the origin to insertion point, can be calculated for the segmented muscle or muscle group within the segmented patient image at a pre-set number of curved or linear intervals. Alternatively, one single segmented muscle can be represented by multiple paths (if, for example, the segmented muscle is fan-shaped). In these cases, the paths can be centrelines of partitions of the muscle volume.

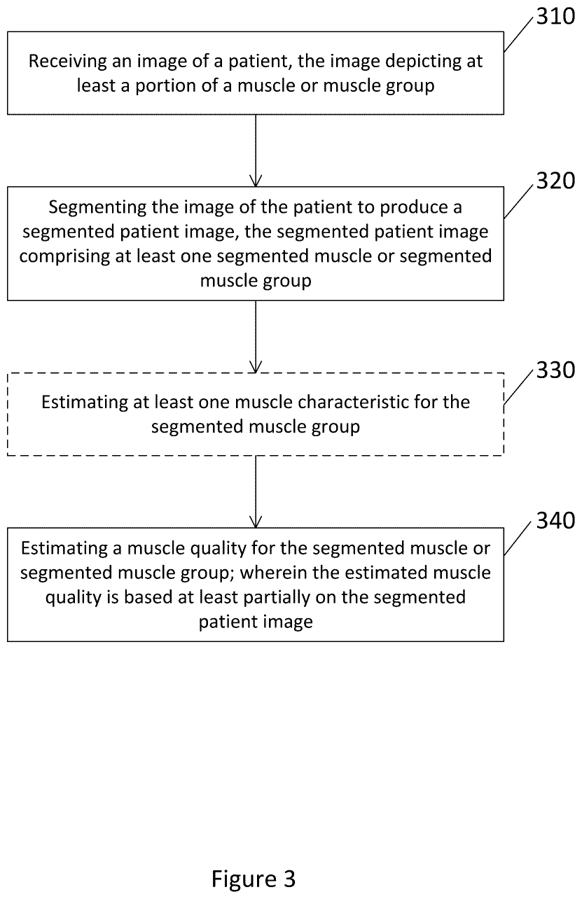

In a still further example, an electronic computing device can be used to additionally or alternatively estimate a muscle quality of a muscle or muscle group. The estimated quality can be used, for example, to rapidly advise a surgeon on surgical decision making such as their approach, implant selection, and implant placement in a pre-operative or intra-operative context. The estimated quality can also be used post-operatively to e.g. guide rehabilitation exercises for a patient. To this end, FIG. 3 depicts an example method implemented on an electronic computing device for estimating a muscle quality of a muscle or muscle group.

An image of a patient is received at 310. The image depicts at least a portion of a muscle or muscle group. The image is then segmented at 320 to produce a segmented patient image. The segmented patient image comprises at least one segmented muscle or segmented muscle group. In some examples, at least one muscle characteristic can be estimated at 330 for the segmented muscle or segmented muscle group. A muscle quality for the segmented muscle or segmented muscle group is then estimated at 340. The estimated muscle quality is based at least partially on the segmented patient image. If a muscle characteristic is estimated at 330, then the muscle quality estimated at 340 can also be based at least partially on the muscle characteristic estimated at 330.

In some examples, the image of the patient received at 310 can substantially be an image as has been described with respect to 110 of FIG. 1B. In some particular examples, the image received at 310 can comprise a CT image or CT scan. The CT image or scan can comprise a plurality of pixels or voxels that have an intensity corresponding to a Hounsfield unit value on the Hounsfield scale. The Hounsfield unit value of a given pixel or voxel corresponds to a radiodensity of an imaged material. As different tissue compositions have different radiodensities, different tissues can appear on the CT image/scan as pixels/voxels of different intensities/Hounsfield unit values. This can be used to distinguish different tissue types within the image received at 310.

In some examples, the image can be segmented at 320 in substantially the same way as described as 120 with respect to FIG. 1B. In examples where the image received at 310 comprises a CT image or CT scan comprising pixels or voxels having an intensity corresponding to a Hounsfield unit value, segmenting the image at 320 can comprise identifying a plurality of pixels or voxels at least partially depicting a muscle or muscle group of the patient, with each pixel or voxel having an intensity corresponding to a Hounsfield unit value, such that the segmented patient image produced at 320 preserves the Hounsfield unit information within the image received at 310. The segmentation can be performed e.g. using an algorithm and/or machine learning model as described as 120 with respect to FIG. 1B.

Examples of muscle characteristics that can optionally be estimated at 330 can include muscle volume, pennation angle, and/or muscle shape. These can be estimated in substantially the same way as described at 130 with respect to FIG. 1B. These can be expressed using e.g. quantitative numbers and/or may be expressed in terms of statistical measurements, such as Z-scores.

In some examples, the muscle quality can be estimated at 340 using a machine learning model (e.g. a CNN) that takes the segmented patient image produced at 320 as an input. The machine learning model can be trained on a dataset of imaged muscles that are labelled with a quality score, which may be quantitative or qualitative. The machine learning model can also take any muscle characteristics estimated at 330 as additional inputs for the determination of the muscle quality at 340.

In examples where the image received at 310 and segmented at comprises pixels or voxels having an intensity corresponding to a Hounsfield unit value, the estimation of muscle quality at 340 can be based at least partially on the Hounsfield unit values of the plurality of pixels or voxels depicting the segmented muscle or segmented muscle group of the patient within the segmented patient image. In some examples, these values may be provided alongside the pixel/voxel data as e.g. metadata or an additional dimension for each pixel/voxel. In other examples, the Hounsfield unit values can be used to identify the extent or volume of different tissues within the segmented muscle or muscle group.

In some examples, estimating the muscle quality at 340 can comprise estimating an extent of fatty infiltration within the segmented muscle or segmented muscle group, with the estimated muscle quality based at least partially on the estimated extent of fatty infiltration. Additionally or alternatively, estimating the muscle quality at 340 can comprise estimating an extent of connective tissue infiltration within the segmented muscle or segmented muscle volume, with the estimated muscle quality based at least partially on the estimated extent of connective tissue infiltration. Other non-muscle tissues, such as scar tissue, can also be identified.

In some examples, the fatty tissue, connective tissue, or scar tissue infiltrating the muscle can be identified by segmentation (either at 320 or in a subsequent segmentation step). For example, different tissues can be segmented based on their intensities (corresponding to a Hounsfield unit value) using e.g. a graph cut algorithm. Alternatively, a machine learning model can be trained to identify fatty tissue and/or connective tissue and/or scar tissue and to discern these tissues from skeletal muscle, using e.g. a training dataset of labelled CT images or scans including Hounsfield unit value information. Once the tissues have been identified within the segmented patient image, the extent of infiltration can be estimated by e.g. determining the depth at which fatty/connective tissue can be found within the muscle, and/or by determining a ratio (e.g. a volumetric ratio) of fatty tissue or connective tissue to skeletal muscle.

The quality for the muscle can then be estimated at 340 by e.g. comparing the extent of fatty infiltration, the extent of connective tissue infiltration, the extent of scar tissue, and/or volumetric ratios of fatty/connective tissue to skeletal muscle and then comparing these measurements with statistical measurements of a population. For example, the determined extent or volumetric ratio of these tissues can be assigned a Z-score, and the overall quality of the muscle estimated at 340 can be based on these Z-scores. In other examples, the determined extent or volumetric ratio can be compared against an ideal muscle used as a reference, with the total score being determined by how the volumetric ratios deviate from the ideal. Similarly, if a muscle characteristic is estimated at 330, then a Z-score for the muscle characteristic (e.g. muscle volume) can be determined and also used to estimate the muscle quality at 340.

In some examples, the muscle quality can be estimated at 340 using an equation or algorithm that weights different muscle characteristics expressed as a Z-score. Alternatively, the Z-scores for different muscle characteristics can be provided to a machine learning model that is trained using supervised or unsupervised learning.

The quality estimated at 340 can be quantitative or qualitative. For instance, the quality can be graded qualitatively. For example, the quality estimated at 340 can may be ‘significantly above average’, ‘above average’, ‘average’, ‘below average’, or ‘significantly below average’. For instance, if Z-scores of muscle characteristics are determined, then a combined Z-score can be compared to an overall population. The individual Z-scores can be weighted depending on e.g. a particular biomechanical task at hand, such as walking or a sit-to-stand transition. In other examples, a machine learning model can be trained to output a numeric result given the segmented patient image and optional muscle characteristics. For instance, a machine learning model can output a numeric value using a sigmoid function. The numerical value can then optionally be translated to a qualitative quality based on e.g. how the output of the sigmoid function compares to a population average.

In some examples, the muscle quality estimated at 340 can be binary, e.g. ‘acceptable’ or ‘unacceptable’. This can be useful in the context of a proposed surgery. For example, the estimation of muscle quality at 340 can be determined using an equation or algorithm that sums up weighted Z-scores representing different characteristics of the muscle. If the sum is below a certain threshold, then the muscle quality may be unacceptable. In other examples, a quantified output from a machine learning model (such as the sigmoid output discussed above) can be combined with a classifier, such as a linear classifier, to determine if the estimated quality is acceptable or unacceptable.

In still further examples, the muscle quality can be estimated at 340 using a machine learning model that accepts the segmented patient image produced at 320 and is trained via unsupervised learning. For instance, a training data set can comprise images of muscles that have been acquired previously, some of which had positive surgical outcomes and others of which had negative surgical outcomes. The machine learning model can used unsupervised learning, such as k-means clustering, to identify groups of muscles based on their quality. The machine learning model can then place the segmented muscle depicted in segmented patient image 120 within one of these groups. In other examples, a similar approach can be taken using a supervised learning approach, with the training image dataset being labelled with surgical outcomes. The machine learning model can then estimate a surgical outcome (e.g. ‘acceptable’ or ‘unacceptable’) for the segmented muscle within the segmented patient image.

In examples where the muscle quality estimated at 340 is a binary qualitative value representing an acceptable or unacceptable muscle, the estimated quality can comprise sub-qualities or sub-scores that allow the user (e.g. a surgeon) to learn more about why the muscle is considered to be acceptable or unacceptable. For example, the quality estimated at 340 can suggest that the muscle will not be suitable for a given surgery, and this can be provided to the surgeon for rapid decision-making. However, the surgeon may wish for more granular information in order to understand why the muscle has an unacceptable quality. The surgeon can then request to review the sub-qualities or sub-scores which could suggest, for example, that while the muscle volume and force production of the muscle are acceptable, the patient's biomechanics are impaired due to presence of connective tissue within the muscle.

In some examples, the muscle quality estimated at 340 can be used as a ‘traffic light’ to guide surgeons and help inform decision making. If the quality is above a certain threshold or is acceptable, the surgeon can proceed with a pre-operative plan without undue concern. If the quality is borderline, the surgeon may need to refine a pre-operative plan and/or use more sophisticated methods, such as 3D pre-operative templating, to account for increased risks. If the quality is unacceptable, the surgeon can rapidly know that the muscle is unsuitable for surgery or will require a different pre-operative approach, and further has the option to investigate deeper in order to understand why.

### General Soft-Tissue Structures

In addition to estimating the force potential, pathing, and/or quality of muscles specifically, the computer implemented methods disclosed herein can additionally or alternatively be used to estimate other properties of muscles. Furthermore, the computer implemented methods disclosed herein can be used to estimate other properties of other soft-tissue structures in general. These soft-tissue structures include muscles and muscle groups in addition to other structures such as joint capsules, ligaments, and/or ligament groups amongst others. The soft-tissue structures assessed in the computer implemented methods disclosed herein can also include a plurality of soft-tissue sub-structures and/or all the soft-tissue structures relevant to a joint (e.g. all soft-tissue structures relevant to a hip joint).

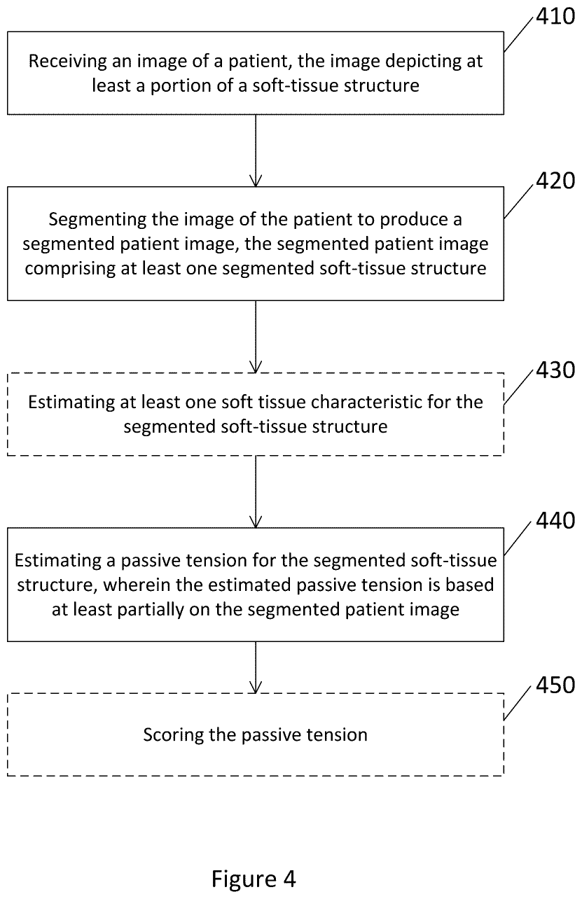

For example, FIG. 4 depicts an example method implemented on an electronic computing device for estimating a passive tension of a soft-tissue structure. An image of a patient is received at 410. The image depicts at least a portion of a soft-tissue structure. The image is then segmented at 420 to produce a segmented patient image. The segmented patient image comprises at least one segmented soft-tissue structure. In some examples, at least soft tissue characteristic can be estimated at 430 for the segmented soft-tissue structure, based on the segmented patient image. A passive tension is then estimated at 440 for the segmented soft-tissue structure. The estimated passive tension is based at least partially on the segmented patient image. If a soft tissue characteristic is estimated at 430, then the passive tension estimated at 440 can also be based at least partially on the soft tissue characteristic estimated at 430. The passive tension estimated at 440 can optionally be scored at 450.

The image of the patient that is received at 410 can substantially be an image as has been described with respect to FIG. 1B. The imaging modality used to acquire the image received at 410 may vary depending on the exact type of soft-tissue structure that is being imaged. For instance, where the soft-tissue structure is a joint capsule, the image received at 410 may be an MRI image to ensure that the different tissues within the joint capsule can be individually distinguished from one another. In other examples, the image received at 410 may be an image that is not traditionally associated with soft tissue imaging, as an X-ray as described herein. Similarly, the image can be segmented at 410 to produce a segmented patient image in substantially the way as 120 with respect to FIG. 1B.

In examples where a soft tissue characteristic is estimated at 430, the soft tissue characteristic can depend on the nature of the segmented soft-tissue structure within the segmented patient image. In examples where the soft-tissue structure is a muscle, the soft tissue characteristic estimated at 430 can include one or more of:
a length of the muscle
an amount of connective tissue within the muscle
a fraction of connective tissue volume within the muscle volume
a material property (e.g. stiffness) of connective tissue relative to the muscle volume
a spatial distribution and/or density of connective tissue (e.g. whether the connective tissue presents as a sheet, a lump, or a diffuse network)
a volume of a tendon associated with the muscle
a cross-sectional area of a tendon associated with the muscle

In examples where the soft-tissue structure is a joint capsule and/or a ligament, the soft tissue characteristic estimated at 430 can include one or more of:
a bulk volume of the structure
a shape of the structure
an average thickness of the structure
a thickness distribution of the structure
an image intensity of the structure (for example, to discern scar tissue)
a texture of the image

In some examples, the soft tissue characteristic estimated at 430 can be a product or ratio of one or more individual soft tissue characteristics. For instance, if the soft-tissue structure is a muscle, the soft tissue characteristic estimated at 430 can include a fraction of the connective tissue volume within the muscle volume. This can be estimated at 430 by estimating a volume of connective tissue, estimating a net muscle volume, and then determining the ratio of the volume of connective tissue to the net muscle volume.

As with respect to the estimation 130 of FIG. 1B, in examples where a soft tissue characteristic is estimated at 430 for the segmented soft-tissue structure, the soft tissue characteristic can be estimated using a trained machine learning model, without using a machine learning model, or by using one or more algorithms in conjunction with a machine learning model. Statistical shape models can also be used to estimate e.g. volumes and shapes of muscles and/or connective tissues based on landmarks derived from the segmented patient image, as described with respect to 130 of FIG. 1B.

As with respect to the estimation 140 of FIG. 1B, the passive tension can be estimated at 440 using a machine learning model which receives the segmented patient image as an input. For example, the machine learning model can be trained using a dataset of segmented images depicting soft-tissue structures that have been labelled with empirically derived or virtually simulated passive tensions. In examples where one or more soft-tissue characteristics are estimated at 430, the passive tension can be estimated at 440 using a machine learning model that also uses muscle characteristics as inputs. In still further examples, the passive tension can be estimated at 440 without the use of a machine learning model, using e.g. a Hill-type representation (if the soft-tissue structure is a muscle) and/or statistical modelling given the soft tissue characteristics estimated at 430.

The optional scoring of the passive tension at 450 can also be determined using statistical modelling, by e.g. expressing the estimated passive tension relative to the mean of a representative population and assigning the estimated passive tension a Z-score. The score can be expressed quantitatively or qualitatively, and/or can be referenced against a context-dependent standard, such as a biomechanical task involving the use of the soft-tissue structure, or in the context of a proposed surgery.

The ability to rapidly and non-invasively estimate the passive tension of soft-tissue structures (and/or force potentials of muscles) from patient images can allow surgeons to develop or modify pre-operative plans for the patient. For example, a surgeon can quickly estimate the passive tension of a soft-tissue structure using a pre-operative image of the patient—which may be taken as part of a routine pre—operative process—in conjunction with a computer implemented method as disclosed herein. The surgeon can then develop a pre-operative plan accounting for the patient-specific passive tension of the soft-tissue structure as estimated from the pre-operative image. Additionally or alternatively, the computer implemented methods disclosed herein can allow a surgeon to propose a change or modification to a pre-operative plan and to assess the impact of that change or modification. The computer implemented methods disclosed herein can further be used to estimate post-operative changes to characteristics of soft tissues due to e.g. proposed changes to the geometry of the soft-tissue structure in question.

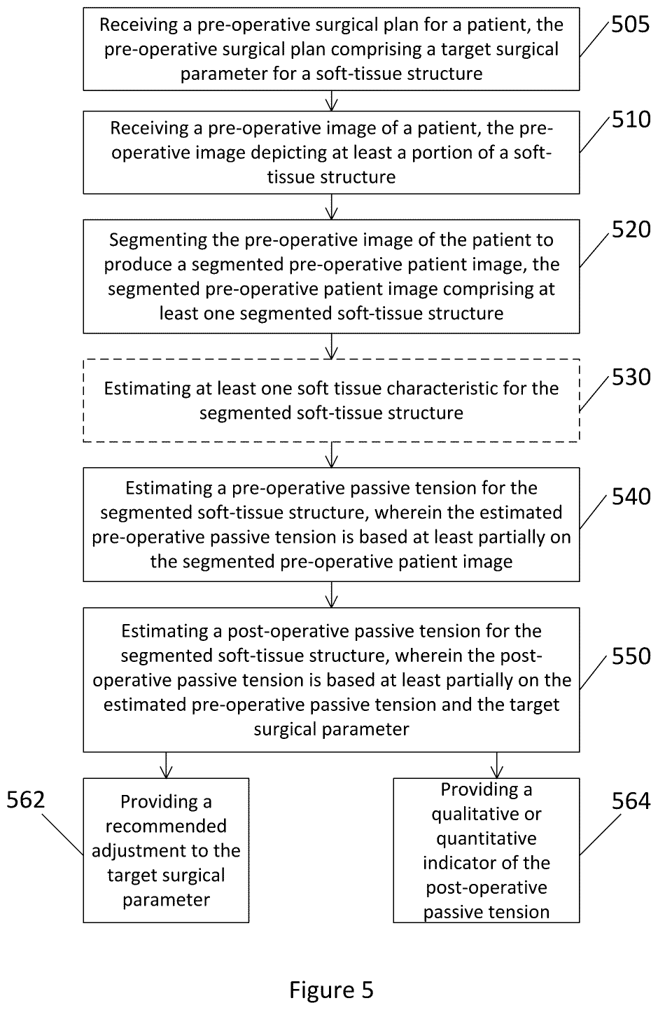

For instance, FIG. 5 depicts an example method implemented on an electronic computing device for estimating a post-operative passive tension of a soft-tissue structure. A pre-operative surgical plan for the patient is received at 505 and comprises a target surgical parameter for a soft-tissue structure. A pre-operative image of the patient is also received at 510 and depicts at least a portion of the soft-tissue structure. The pre-operative image is then segmented at 520 to produce a segmented pre-operative patient image. The segmented pre-operative patient image comprises at least one segmented soft-tissue structure. In some examples, a soft tissue characteristic can optionally be estimated at 530 for the segmented soft-tissue structure based on the segmented pre-operative patient image, substantially as described herein with respect to 430 of FIG. 4. A pre-operative passive tension is then estimated at 540 for the segmented soft-tissue structure. The estimated pre-operative passive tension is based at least partially on the segmented pre-operative patient image and can be estimated substantially as described herein with respect to 440 of FIG. 4. If a soft tissue characteristic is estimated at 530, then the pre-operative passive tension estimated at 540 can also be based at least partially on the soft tissue characteristic estimated at 530. A post-operative passive tension for the segmented soft-tissue structure is then estimated at 550. The estimated post-operative passive tension is based at least partially on the pre-operative passive tension estimated at 540 and the target surgical parameter received at 505.

In some examples, the post-operative passive tension can be estimated at 550 using one or more machine learning models. For instance, in some examples, a generative machine learning model can be used to generate an image or feature map that represents the segmented soft-tissue structure if pre-operative soft-tissue structure were modified by the target surgical parameter received at 505. For instance, if the target surgical parameter corresponds to a shortening in the length of a muscle, the generative machine learning model can be trained to produce an image showing (or feature map corresponding to) the pre-operative soft-tissue structure with a shortened muscle. The image or feature map produced by the generative machine learning model can then be treated as a patient image and its associated passive tension can be estimated substantially in the same way as at 440 with respect to FIG. 4. An example machine learning model can be trained by constructing a virtual model of a joint comprising rigid structures (such as bones and implants) and non-rigid structures (such as muscles.) The virtual model can then be perturbed many times based on surgical parameters, such as implant orientations/positions, and can then be used to mechanistically generate multiple possible post-operative states. A machine learning model can then be trained with the initial state, a given perturbation, and an associated post-operative state as a training dataset. Once trained, the machine learning model can generate the post-operative state directly from an initial state and a perturbation as an input.

In still further examples, estimating the post-operative passive tension at 550 can comprise constructing a post-operative virtual model based at least partially on the segmented pre-operative patient image and target surgical parameter. For example, a pre-operative virtual model comprising one or more pre-operative bone positions and orientations (or positions/orientations of other anatomical structures) can be determined from the pre-operative image received at 510, through e.g. fitting SSMs to the pre-operative anatomical features depicted in the pre-operative patient image. New bone positions and orientations (or positions/orientations of other anatomical structures) can then be determined corresponding to anatomical changes related to the target surgical parameter received at 505—in addition to other geometric changes, such as new joint centres/foci (e.g. in the case of a knee)—to determine a post-operative virtual model.

In some examples, a generative machine learning model can be used to construct an image or feature map based on the post-operative virtual model. The image or feature map produced by the generative machine learning model can then be treated as a patient image and its associated passive tension can be estimated substantially in the same way as at 440 with respect to FIG. 4.

In still further examples, the post-operative virtual model can undergo a simulation to estimate the post-operative passive tension at 550. For instance, a pre-defined model of muscle trajectory can be used to update the post-operative virtual model with insertion and origin points. This can be based on a patient-specific bony anatomy (e.g. derived from patient-specific pre-operative images) or e.g. inferred using SSMs. Muscle trajectories can also be determined based on the patient's bulk anatomy (e.g. derived from patient-specific pre-operative images) or e.g. inferred using SSMs. Changes in the length of the soft-tissue structure, or other soft tissue characteristics, can then be calculated by simulating motion through one or more ranges of motion (e.g. a sit-to-stand motion) for the post-operative virtual model. The changes in length (or other soft tissue characteristic) at each discrete point in motion or in time in the simulation, in conjunction with a constitutive model relevant to the soft-tissue structure, can then be used to estimate a post-operative passive tension for the soft-tissue structure. For example, the constitutive model can comprise an elastic model, a neo-Hookean model, or a hyperelastic model amongst others.

In some further examples, a recommended adjustment to the target surgical parameter can be provided at 562 based at least partially on the post-operative passive tension estimated at 550. For instance, if the proposed target surgical parameter would result in an unacceptable post-operative passive tension for the soft-tissue structure, then the electronic computing device can recommend an adjusted surgical parameter to avoid the unacceptable outcome.

Additionally or alternatively, the electronic computing device can provide a qualitative or quantitative indicator of the post-operative passive tension to advise the surgeon at 564. For example, although the post-operative passive tension estimated at 550 can be numerical and in absolute units, this may be difficult for the surgeon to contextualise. Providing a qualitative indicator of the post-operative passive tension can allow the surgeon to make a rapid and informed decision as to whether the target surgical parameter is fit for purpose, or whether an adjustment is required.

For example, the electronic computing device can warn the surgeon at 564 that the estimated post-operative passive tension goes beyond known physiological limits and/or is associated with a risk. Additionally or alternatively, the surgeon can be warned when a muscle is slack at some point in a range of motion associated with the soft-tissue structure, when it is known that the muscle is required to be producing a force at that point. This is particularly relevant when a post-operative virtual model is simulated in order to estimate the post-operative passive tension at 550. In still further examples, the surgeon can be notified that the target surgical parameter is within an acceptance zone when the estimated post-operative passive tension is within the bounds of the contralateral side. In still further examples, the electronic computing device can provide a quantitative change indication represented as a percentage, with warnings at fixed limits or at limits defined by the surgeon, such as 5%, 10%, etc.

In further examples, the method can further comprise receiving an adjustment to the target surgical parameter received at 505. For instance, the surgeon may make an adjustment to the target surgical parameter based on the post-operative passive tension estimated at 550, the recommended adjusted target surgical parameter recommended at 562, and/or feedback provided at 564. An adjusted post-operative passive tension for the segmented soft-tissue structure can then be estimated based at least partially on the adjustment to the target surgical parameter as has been described herein.

In some examples, a post-operative passive tension can be estimated at 550 and the surgeon provided with a quantified change in the passive tension of the soft-tissue structure (i.e. a difference between the estimated pre-operative passive tension and estimated post-operative) at 564. The surgeon can then adjust the target surgical parameter and can receive an adjusted post-operative passive tension for the soft-tissue structure. The surgeon can then iteratively adjust the target surgical parameter (or set of parameters) and iteratively receive the adjusted post-operative passive tension corresponding to those adjustments. The surgeon can then select a final surgical parameter based on what they consider to be an ideal plan given the estimated change in passive tension.

The target surgical parameter that is received at 505 can depend on the soft-tissue structures involved in the pre-operative plan received at 510 and the nature of the procedure. For instance, the pre-operative plan can correspond to a joint replacement surgery, such as a total hip arthroplasty or a knee arthroplasty. The target surgical parameter received at 505 can comprise one or more of:
a target joint offset
a target length adjustment
an acetabular cup position
an acetabular cup orientation (e.g. inclination and anteversion angles) a stem size
a stem portion
a stem orientation (e.g. anteversion, varus/valgus, and/or flexion/extension) a location of a femoral neck resection
a muscle on which to perform a muscle release
an amount of muscle release performed on a release
a location of a capsule to be cut
a location of a capsule to be removed
a tibial tray size
a tibial tray position
a tibial tray orientation
a tibial spacer thickness
a femoral component size
a femoral component position
a femoral component orientation.

It will be apparent that the above target surgical parameters are only examples, and other target surgical parameters can be received with the pre-operative plan at 505. Similarly, it will be apparent that the pre-operative plan received at 505 can correspond to a joint replacement surgery other than a total hip arthroplasty or a knee arthroplasty. In other examples, the pre-operative plan can correlate to other joint arthroplasties. In still further examples, the pre-operative plan can correlate to a surgery other than a joint arthroplasty.

In addition to receiving feedback on (and possibly adjustments relating to) a target surgical parameter and/or pre-operative plan, in some examples, it can be advantageous to first estimate a pre-operative passive tension for a soft-tissue structure, and then to determine a post-operative plan and/or target surgical parameter on the basis of the estimated pre-operative passive tension.

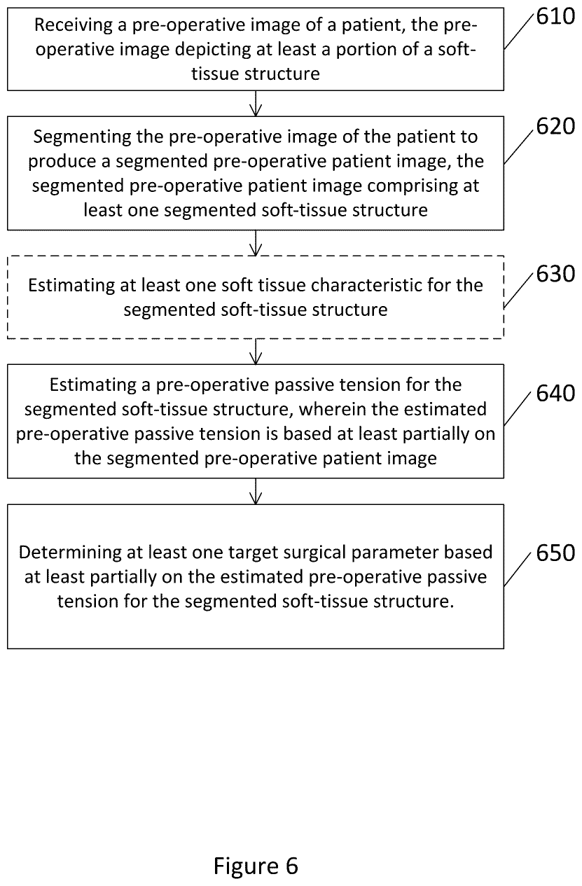

To this end, FIG. 6 depicts an example method implemented on an electronic computing device for developing a pre-operative plan for a patient. A pre-operative image of the patient is received at 610 depicting at least a portion of the soft-tissue structure. The pre-operative image is then segmented at 620 to produce a segmented pre-operative patient image. The segmented pre-operative patient image comprises at least one segmented soft-tissue structure. In some examples, a soft tissue characteristic can optionally be estimated at 630 for the segmented soft-tissue structure based on the segmented pre-operative patient image, substantially as described herein with respect to 430 of FIG. 4. A pre-operative passive tension is then estimated at 640 for the segmented soft-tissue structure. The estimated pre-operative passive tension is based at least partially on the segmented pre-operative patient image and can be estimated substantially as described herein with respect to 440 of FIG. 4. If a soft tissue characteristic is estimated at 630, then the pre-operative passive tension estimated at 640 can also be based at least partially on the soft tissue characteristic estimated at 630. At least one target surgical parameter is then determined at 650 based at least partially on the pre-operative passive tension estimated at 640 for the segmented soft-tissue structure.

In some examples, the target surgical parameter determined at 650 can be such that an estimated post-operative passive tension for the segmented soft-tissue structure is substantially the same as the pre-operative passive tension estimated at 640. For example, the electronic computing device can numerically iterate through a range of values of the target surgical parameter, with an estimated post-operative passive tension being calculated substantially as described at 550 with respect to FIG. 5. Additionally or alternatively, the electronic computing device can consult a pre-populated lookup table to find an initial condition from which to iterate, or to find a target surgical parameter that satisfies the required post-operative passive tension.

In still further examples, the target surgical parameter determined at 650 can be such that the estimated post-operative passive tension is substantially the same as the passive tension of the soft-tissue structure on the contralateral side. In still further examples, and particularly in the case of arthroplasty, the target surgical parameter determined at 650 can be such that the post-operative passive tension of the soft-tissue structure does not result in dislocating forces on the replacement joint through a range of motion. Such forces may be stored as absolute thresholds or may be determined e.g. in a patient-specific manner using machine learning and/or the construction of a virtual model of the relevant joint. In still further examples, the target surgical parameter determined at 650 can be such that the post-operative passive tension does not cause bony impingement or soft-tissue impingement on the joint through a range of motion, which can also be determined through e.g. simulation of a post-operative virtual model.

In still further examples, the target surgical parameter determined at 650 can be such that the resulting post-operative passive tension (which can be estimated e.g. as substantially described at 550 with respect to FIG. 5) is within a range of quantified change from the pre-operative passive tension estimated at 640. For example, the resulting post-operative passive tension can be within an arbitrary percentage or an absolute change as defined by the surgeon (e.g. a 5% increase in passive tension.) For example, the surgeon can specify the desired change in the passive tension, and the electronic computing device can then iterate to numerically determine a target surgical parameter that corresponds to the specified desired change.

In still further examples, the target surgical parameter determined at 650 can be such that the post-operative passive tension of the soft-tissue structure balances an active muscle force generation and/or joint capacity. The joint capacity can be determined using statistical shape modelling in combination with patient-specific imaging. This can prevent the target surgical parameter from resulting in an overly tense post-operative joint that can likely reduce active force production past some point.

In still further examples, the target surgical parameter determined at 650 can be such that the resulting post-operative passive tension is substantially the same as an ideal tension estimated for the patient based at least partially on one or more of their demographics data, pre-operative functional data, pre-operative anatomical data, and/or lifestyle. For example, the ideal tension can be determined using statistical modelling of e.g. passive tensions related to undesired outcomes for a cohort representative of the patient. The determination of the ideal passive tension can additionally or alternatively be determined using machine learning or other artificial intelligence.

Furthermore, it will be clear that although the methods described herein (examples of which are provided in FIG. 5 and FIG. 6) are framed in terms of pre-operative and post-operative passive tension, other kinds of soft tissue traits and/or tensions can also be estimated. For instance, instead of estimating a pre-operative and/or post-operative passive tension using the computer-implemented methods disclosed herein, a surgeon can alternatively estimate a pre-operative and/or post-operative active tension for the soft-tissue structure.

### Radiographic Images and Soft-Tissue Structures

Conventional radiographic images (e.g. X-rays) are often routinely taken during pre-operative and post-operative consultations between surgeons and patients, and can offer a convenient source of patient-specific information without an increase in ordinary clinical burden. However, it is known that extracting soft-tissue information from X-rays and other conventional radiographic images can be difficult due to insufficient tissue contrast within the image. For instance, although bony structures can be very visible on an X-ray, soft tissues such as muscles, tendons, and other connective tissues may not be easily seen. Even if the soft tissues can be seen within the X-ray, it can be difficult to distinguish one soft tissue from another. This can pose a problem when trying to e.g. differentiate muscle from connective tissues.

The Applicant has found that despite these shortcomings, machine learning models can be trained to extract soft tissue information from X-rays and other radiographic images.

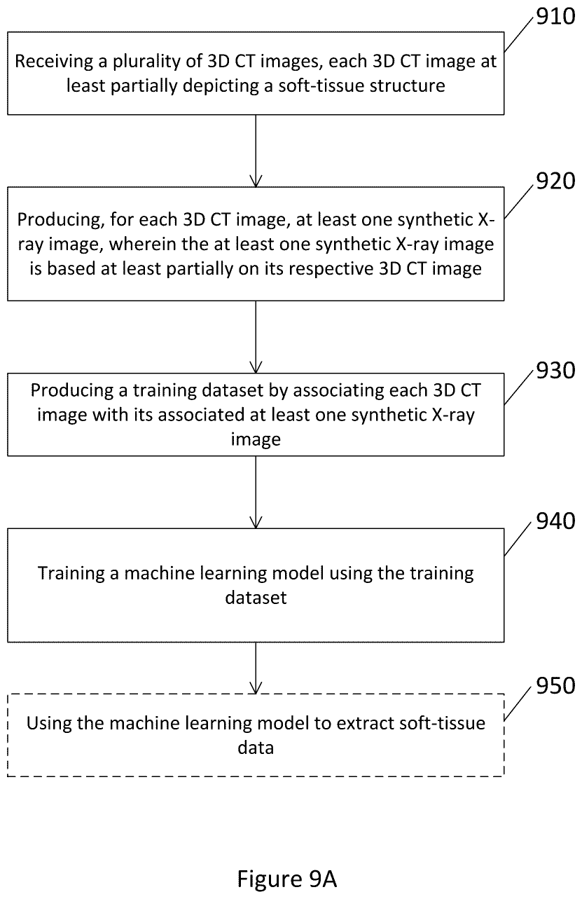

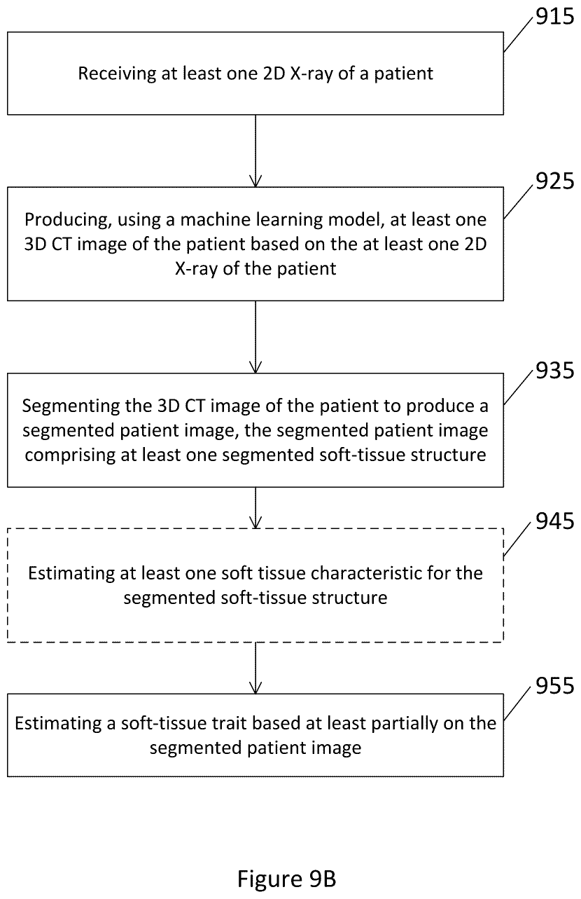

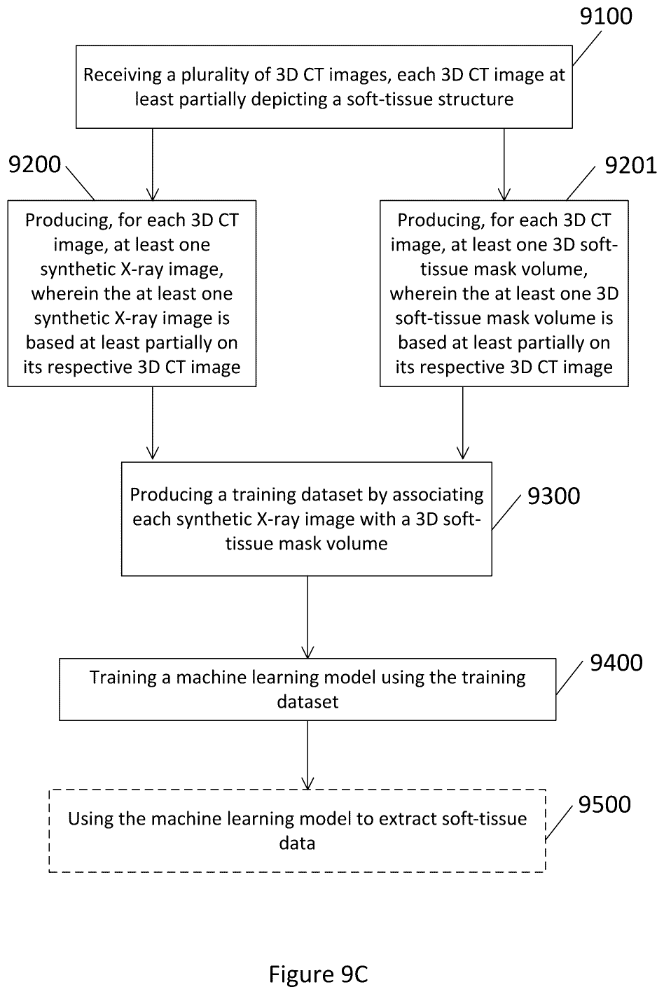

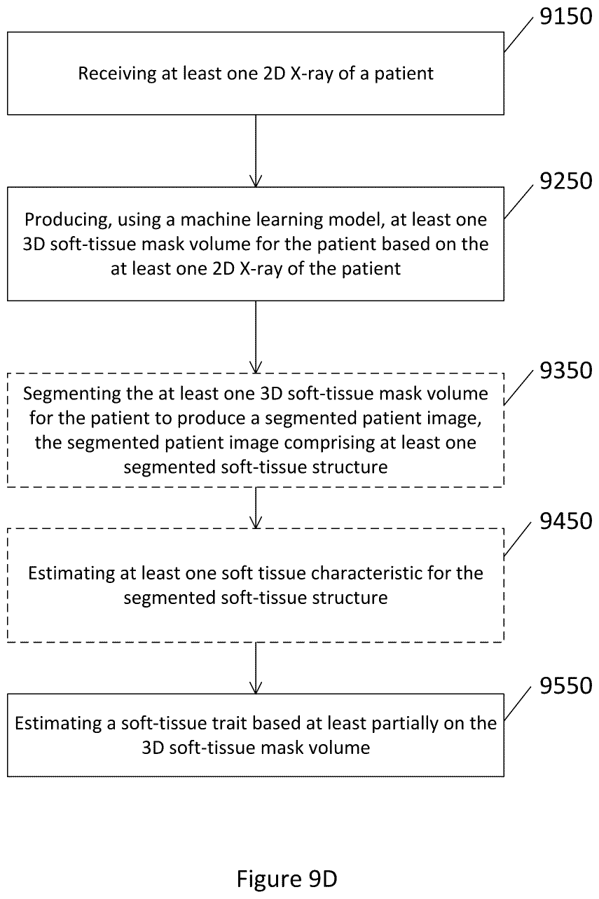

FIG. 9A depicts an example method for training a machine learning model. A plurality of 3D CT images are received at 910. Each 3D CT image depicts at least one soft-tissue structure. At least one synthetic X-ray image is produced for each 3D CT image at 920, wherein the at least one synthetic X-ray image is based on its respective 3D CT image. A training dataset is then produced at 930 by associating each 3D CT image with its associated at least one synthetic X-ray. A machine learning model is then trained using the training dataset at 940. The machine learning model can optionally be used to estimate a soft-tissue trait at 950.

In some examples, the 3D CT images that are received at 910 are clinical images that have been acquired from real-world subjects. The 3D CT images can be acquired from a wide range of different people or across a sufficiently broad cohort to reduce the likelihood of overfitting or underfitting. For instance, subjects of different ages, genders, heights, and weights can be imaged to produce a diverse training dataset of 3D CT images. In other examples, the 3D CT images that are received at 910 can be acquired from a relatively narrow cohort of subjects if a machine learning model will be trained to extract soft tissue structures from a specific group of people. In these instances, multiple machine learning models trained for different cohorts of individuals may by trained and used.

In other examples, the 3D CT images that are received at 910 can be synthetic 3D CT images. For example, the 3D CT images can comprise 3D CT images produced by a machine learning model, such as a generative adversarial network configured to produce synthetic 3D CT images. In other examples, the 3D CT images that are received at 910 can comprise synthetic 3D CT images that are produced e.g. by simulating a 3D CT scanning process on a virtual model. In still further examples, the 3D CT images that are received at 910 can comprise 3D CT images that are taken of synthetic models, such as a CT phantom.

In some examples, the synthetic 2D X-ray images that are produced at 920 can be produced using a trained machine learning model. For example, a generative machine learning model (such as a generative adversarial network) can be trained on 3D CT images of subjects that have been labelled with real-world X-ray images of the subject. The machine learning model can then be trained to synthesise an X-ray given a CT image as input. In other examples, the synthetic X-ray images produced at 920 can be created by simulation (by e.g. simulating the constituent materials, their X-ray properties, and then modelling X-ray wave propagation through the simulated materials by e.g. ray tracing).

In some examples, the machine learning model trained at 940 can be trained using deep learning. In some examples, the machine learning model can be a deep neural network (DNN). In some examples, the machine learning model can be trained using additional data. For instance, in addition to the labelled 3D CT images and synthetic 2D X-ray images produced at 920, the dataset used to train the machine learning model can also include metadata such as age, sex, height, weight, ethnicity, or other patient-specific factors of the imaged subject.

The machine learning model trained at 940 can then be used (at e.g. 950) to produce synthetic 3D CT images given 2D X-rays as inputs. Soft-tissue data can be extracted from the produced 3D CT image using, for example, the segmentation techniques to produce a segmented patient image as discussed herein, and the disclosed techniques used to estimate soft-tissue traits therefrom. In some examples, the inputs for the machine learning model trained at 940 can further comprise patient-specific information such as the age, sex, height, weight, and/or ethnicity if the training data includes corresponding patient-specific information.

FIG. 9B depicts an example method of extracting soft-tissue data using such a machine learning model. At least one 2D X-ray image of a patient is received at 915. A machine learning model is used to produce at least one 3D CT image of the patient at 925 based at least partially on the at least one 2D X-ray image. The at least one 3D CT image depicts at least a portion of a soft-tissue structure. The at least one 3D CT image is segmented at 935 to produce a segmented patient image, the segment patient image comprising at least one segmented soft-tissue structure. A soft-tissue trait (e.g. a muscle force capacity, a quality, and/or a passive or active tension) is then estimated at 955 based at least partially on the segmented patient image. A soft-tissue characteristic can optionally be estimated at 945, in which case the soft-tissue trait is based at least partially on the soft-tissue characteristic.

The machine learning model used at 925 can be as described above. The 3D CT image can be segmented using any of the techniques described herein. Similarly, the soft-tissue trait and/or soft-tissue characteristic can be estimated using any of the techniques described herein.

FIG. 9C depicts another example method for training a machine learning model. A plurality of 3D CT images are received at 9100. Each 3D CT image depicts at least one soft-tissue structure. At least one synthetic X-ray image is produced for each 3D CT image at 9200, wherein the at least one synthetic X-ray image is based on its respective 3D CT image. At least one 3D soft-tissue mask volume is also produced for each 3D CT image at 9201, wherein the at least one 3D soft-tissue mask volume is based on its respective 3D CT image. The 3D soft-tissue mask image can comprise, for example, a 3D muscle structure mask volume. A 3D bone mask volume can also be produced for each 3D CT image at 9201. A training dataset is then produced at 9300 by associating each at least one 3D soft-tissue mask with the corresponding at least one synthetic X-ray originating from the same CT image. The training dataset can comprise multiple 3D soft-tissue masks associated with a single synthetic X-ray or can comprise a plurality of individual 3D masks associated with a single synthetic X-ray. A machine learning model is then trained using the training dataset at 9400. The machine learning model can optionally be used to estimate a soft-tissue trait at 9500.

As with respect to FIG. 9A, the 3D CT images that are received at 9100 can be substantially the same as at X with respect to 910 of FIG. 9A. Similarly, the synthetic X-rays can be produced at 9200 in substantially the same way as at 920 with respect to FIG. 9A. The 3D mask volumes (e.g. soft-tissue masks and/or bone masks) can be produced at 9201 using segmentation methods as has been described herein. For example, the mask volumes can be determined manually by an operator, identified by a machine learning model, or determined using statistical shape modelling.

The machine learning model trained at 9400 can then be used to 3D soft-tissue mask volumes and/or 3D bone mask volumes given 2D X-rays as inputs. Soft-tissue data can be extracted from the produced 3D mask volume using the techniques disclosed herein. In some examples, the inputs for the machine learning model trained at 9400 can further comprise patient-specific information such as the age, sex, height, weight, and/or ethnicity if the training data includes corresponding patient-specific information.

FIG. 9D depicts an example method of extracting soft-tissue data using such a machine learning model. At least one 2D X-ray image of a patient is received at 9150. A machine learning model is used to produce at least one 3D soft-tissue mask volume of the patient at 9250 based at least partially on the at least one 2D X-ray image. A soft-tissue trait (e.g. a muscle force capacity, a quality, and/or a passive or active tension) is then estimated at 9550 based at least partially on the segmented patient image. A soft-tissue characteristic can optionally be estimated at 9450, in which case the soft-tissue trait is based at least partially on the soft-tissue characteristic. A segmented patient image can also be produced at 9350 based on the soft-tissue mask volume if required.

The machine learning model used at 9250 can be as described above. Similarly, the soft-tissue trait and/or soft-tissue characteristic can be estimated using any of the techniques described herein.

### Configured System

FIG. 7 depicts an example system capable of implementing the methods disclosed herein. The system comprises a client-side application 701 and server 702. The client-side application 701 can be implemented on an electronic computing device such as a mobile phone, laptop, personal device, or other electronic computing device as discussed herein, and can be used by a surgeon, rehabilitation professional, or other user. For instance, client-side application 701 can be implemented on a personal computer in a surgeon's office, and the surgeon can use the client-side application 701 during a pre-operative consultation with a patient. For instance, the client-side application 701 can be implemented on a personal electronic computing device 11 with respect to FIG. 1A. The server backend 702 can be implemented on one or more electronic computing devices such as physical or virtual servers, as discussed herein, such as server 13 with respect to FIG. 1A. Client-side application 701 can communicate with server 702 over a network, such as network 12 with respect to FIG. 1A.

The client-side application 701 can include an image uploading module 705 for uploading clinical images for subsequent segmentation and processing. These images can be e.g. pre-operative or post-operative clinical images such as X-rays or CT scans depicting patient anatomy, as outlined at 110 with respect to FIG. 1B. The uploaded images can then be stored in image database 715 on server 702 and/or may be processed by an image processing application 710. For instance, image processing application 710 can be configured to construct 2.5D images from stereographic pairs of 2D images. Image processing application 710 can use one or more machine learning models which can be e.g. stored in machine learning repository 735.

The client-side application 701 can further include a submission module 720 to allow the user to submit a pre-operative plan. The pre-operative plan can be stored in database 725 of server 702. This can be used when the user wishes, for example, to estimate a post-operative passive tension or other trait of a soft-tissue structure that depends on a pre-operative plan.

Sever 702 implements an image segmentation application 730 to segment the images received from the upload module 705, optionally processed at 710, and/or stored in database 715. The image segmentation application 730 can substantially be implemented as described at 120 with respect to FIG. 1B. As has been described, image segmentation application 730 can utilise a machine learning model that can be stored in a machine learning model repository 735 on server 702. Image segmentation application 730 can also be configured for different forms of image processing (e.g. intensity normalisation) prior to or in conjunction with segmentation.

The server 702 can include an anatomy characterisation application 740 to estimate a soft-tissue characteristic, muscle characteristic, and/or bone characteristic based on the output from the image segmentation application 730. For example, the soft tissue characterisation application 740 can be configured to estimate at least one muscle characteristic, soft-tissue characteristic, and/or bone characteristic as has been described, for instance, with respect to 130 of FIG. 1B, 230 and/or 235 of FIG. 2, 330 of FIG. 3, 430 of FIG. 4, 530 of FIG. 5, and/or 630 of FIG. 6. The anatomy characterisation application 740 can utilise one or more machine learning models as has been described herein, and these can be stored in machine learning model repository 735. The anatomy characterisation application 740 can additionally or alternatively reference one or more statistical shape models which can be stored in anatomical SSM database 745 on server 702. The anatomical SSM database 745 can further comprise other statistical information, such as statistical norms of different attributes (e.g. muscle force potentials) in order to calculate Z-scores and to compare estimated measurements to representative populations.

Client-side application 701 includes a request interface 750 that allows the user to request an estimate of one or more soft tissue traits using the methods disclosed herein. For example, the user can use request interface 750 to request:
an estimate of a post- or pre-operative force production for a soft-tissue structure by making a force request 7501,
an estimate of a post- or -pre-operative passive or active tension for a soft-tissue structure by making a tension request 7502,
a muscle quality score or other trait score (e.g. a force production score) by making a score request 7503,
an estimate of a muscle pathing by making a path request 7504, and/or
a suggested surgical parameter by making a target surgical parameter request 7505.

The request made by the user via request interface 750 can be provided to anatomy characterisation application 740 to determine which characteristics should or can be estimated. For instance, if the request by the user is a force request 7501, then the anatomy characteristic determined by anatomy characterisation application 740 can be a bulk muscle volume as described at 130 with respect to FIG. 1B.

Estimation engine 755 receives the segmented image from image segmentation application 730 (and any characteristics estimated by anatomy characterisation application 740) to process the request made via request interface 750. The estimation engine 755 can include force production estimation module 7551, tension estimation module 7552, score determination module 7553, and pathing estimation module 7554 to estimate soft tissue force productions (e.g. as at 140 of FIG. 1B), passive and active tensions (e.g. as at 440 of FIG. 4), muscle or soft tissue scores or qualities (e.g. as at 340 of FIG. 3), and/or muscle pathing (e.g. as at 240 of FIG. 2). The estimation engine 755 can utilise one or more machine learning models stored in machine learning model repository 735 and/or one or more anatomical SSM models stored in anatomical SSM database 745 to handle the request received from request 43interface 750.

The estimation engine 755 can additionally or alternatively use simulation results derived from virtual simulation engine 760 running on server 702. The virtual simulation engine 760 can be configured to construct virtual models of pre-operative or post-operative patient anatomy and to simulate their motion or biomechanics to assist estimation engine 755. Virtual simulation engine 760 can receive anatomical SSM data from anatomical SSM database 745 and can receive implant model data from implant model database 765 in order to model implants. Virtual simulation engine 760 can also receive pre-operative plan information stored in pre-operative plan database 725 (in order to e.g. simulate post-operative models) and/or can receive images stored in image database 715 and/or segmented by image segmentation application 730.

The server 702 can additionally implement a target surgical parameter determination module 7555 configured to determine a target surgical parameter (e.g. as at 650 of FIG. 6) if a target surgical parameter request 7505 is received by server 702. The target surgical parameter determination module 7555 can communicate at least with virtual simulation engine 760, implant model database 765, and machine learning model repository 735 if required. The target surgical parameter determination module 7555 can also receive an estimation output by estimation engine 755. For instance, if the target surgical parameter request 7505 includes a requirement that the post-operative passive tension of a soft-tissue structure in question remains substantially the same as the pre-operative passive tension, then the estimation engine 755 can first estimate the pre-operative passive tension and then provide the pre-operative passive tension to the target surgical parameter determination module 7555.

Server 702 also comprises a report generation application 770 used to generate reports including, for example, the output of estimation engine 755 and/or target surgical parameter determination module 7555. The report can also comprise other information including, for example, segmented patient images, depictions of the relevant soft-tissue structures, and/or images showing muscle pathing. These depictions can be 2D or 3D. A report download module 775 running on client-side application 701 receives the report generated by report generation application 770. In some examples, the report can be an interactive dashboard that is made available to the client application 701.

The client-side application 701 can further comprise a target surgical parameter adjustment submission module 780 that can be used to submit adjusted target surgical parameters to the server 702 via pre-operative plan submission module 720. For instance, the target surgical parameter adjustment submission module can be used to assess an adjusted pre-operative plan after an initial pre-operative plan has been provided to the server 702 and a request from request interface 750 has been handled.

It will be apparent that the system depicted in FIG. 7 is just one example of a system configured to implement the methods described herein. In other examples, one or more of the applications or modules of the system can be distributed across one or more electronic computing devices. For example, while the server 702 is shown to include databases and repositories, in other examples, databases and repositories may be stored across one or more separate electronic computing devices. Furthermore, the applications hosted by server 702 may be distributed across one or more electronic computing devices. Additionally, while client-side application 701 has been described as a single application comprising several modules, the client-side application 701 can, in some examples, refer to a suite of multiple, separate applications made available to a user.

Furthermore, in some examples, the modules and/or applications operated or hosted by client-side application 701 and/or server 702 may be embodied in physical circuitry. For instance, estimation engine 755 and/or anatomy characterisation application 740 can operate on dedicated circuitry, such as a dedicated GPU. This can be particularly advantageous if a large amount of data needs to be processed in parallel, such as the application of a machine learning model.

### Worked Example

The following worked example illustrates a non-limiting use of the system of FIG. 7 and the methods that can be implemented thereon.

A surgeon consults with a patient before a total hip arthroplasty. The surgeon wishes to estimate a pre-operative passive tension of the patient's gluteus medius, the pathing of the gluteus medius, and an overall score for the gluteus medius. The surgeon submits pre-operative clinical images to image database 715 via image upload module 705 alongside a tension request 7502, pathing request 7504, and score request 7503 via request interface 750. The image is segmented at segmentation application 730 and is provided to anatomy characterisation application 730. Anatomy characterisation application 740 estimates a length of the gluteus medius and a bulk shape of the gluteus medius using a statistical shape model from anatomical SSM database 745 (although in alternative examples, the bulk shape of the gluteus medius may be directly estimated from the segmentation of the muscle). These characteristics are provided to estimation engine 755 and are used as inputs—along with pixel/voxel data from the segmented patient image provided by image segmentation application 730—for multiple machine learning models accessed from repository 735. The pre-operative passive tension, pathing, and overall score for the muscle are estimated by estimation engine 755 and are incorporated into a report generated by report generation application 770.

The surgeon downloads the report using the download module 775. Satisfied with the estimated score and estimated pathing, the surgeon submits a request for a suggested acetabular cup position and acetabular cup orientation via target surgical parameter request module 7505 via request interface 750. The request includes the pre-operative passive tension previously estimated by estimation engine 755 and includes a requirement that the post-operative passive tension of the gluteus medius is substantially the same as its pre-operative passive tension. Target surgical parameter determination module 7555 iterates through a range of possible acetabular cup positions and orientations by modelling and simulating the patient's hip joint post-operatively using virtual simulation engine 760. After a suitable position and orientation is found, a report is generated by report generation application 770 including the suggested surgical parameters and is downloaded by the surgeon.

### Electronic Computing Devices

FIG. 8 depicts a block diagram of an example of an electronic computing device 800. The electronic computing device 800 can correspond to, for example, electronic computing device 11 with respect to FIG. 1B. Similarly, the electronic computing device 800 can correspond to, for example, server 13 with respect to FIG. 1B. The electronic computing device 800 can be configured to run client-side application 701 with respect to FIG. 7, and/or can be configured as server 702 with respect to FIG. 7.

The electronic computing device 800 comprises a CPU (central processing unit) 810, a main memory 820, one or more peripherals 830, and a GPU (graphics processing unit) 850. The CPU 810, main memory 820, peripherals 830, and GPU 840 are connected by a bus 840. In the case of a PCIe (PCI Express) topology, the bus 840 includes a root complex, and each of the peripherals 830 and the GPU 940 are PCIe endpoints. The GPU 850 can communicate with the peripherals 830 directly via the bus 840. In some cases, more than one CPU 810 and/or more than one GPU 850 is provided.

The peripherals 830 may include drives in an array, storage controllers (such as RAID controllers), network controllers (such as network interface cards), switches (such as PCIe switches configured to connect further peripherals), or any variety of devices.

The peripherals 830 and the GPU 850 can have access to the main memory 820 via the bus 840. Through DMA (direct memory access), this can allow the peripheral 830 or the GPU 840 to read data to and write data from the main memory. This can involve copying the data from the main memory 820 to a memory local to the peripheral 830 or the GPU 850.

The disclosed methods may be executed through software stored on non-transitory media that can run on any suitable device. In some examples, the software may be stored on a non-transitory computer-readable storage medium having instructions stored thereon that, when executed by one or more processors of an electronic computing device, performs a method for estimating a force potential of a muscle or muscle group, the method comprising: receiving an image of a patient, the image depicting at least a portion of a muscle or muscle group, segmenting the image of the patient to produce a segmented patient image, the segmented patient image comprising at least one segmented muscle or segmented muscle group, and estimating a muscle force potential for the segmented muscle or segmented muscle group; wherein the estimated muscle force potential is based at least partially on the segmented patient image.

In some examples, software may be stored on a non-transitory computer-readable storage medium having instructions stored thereon that, when executed by one or more processors of an electronic computing device, performs a method for estimating a passive tension of a soft-tissue structure, the method comprising: receiving an image of a patient, the image depicting at least a portion of a soft tissue structure, segmenting the image of the patient to produce a segmented patient image, the segmented patient image comprising at least one segmented soft-tissue structure, and estimating a passive tension for the segmented soft-tissue structure; wherein the estimated passive tension is based at least partially on the segmented patient image.

In some examples, software may be stored on a non-transitory computer-readable storage medium having instructions stored thereon that, when executed by one or more processors of an electronic computing device, performs a method for developing a pre-operative plan for a patient, the method comprising: receiving a pre-operative image of the patient, the image depicting at least a portion of a soft-tissue structure, segmenting the pre-operative image of the patient to produce a segmented pre-operative patient image, the segmented pre-operative patient image comprising at least one segmented soft-tissue structure, estimating a pre-operative passive tension for the segmented soft-tissue structure, wherein the estimated pre-operative passive tension is based at least partially on the segmented pre-operative patient image, and determining at least one target surgical parameter based at least partially on the estimated pre-operative passive tension for the segmented soft-tissue structure.

In some examples, software may be stored on a non-transitory computer-readable storage medium having instructions stored thereon that, when executed by one or more processors of an electronic computing device, performs a method for estimating a post-operative passive tension of a soft-tissue structure, the method comprising: receiving a pre-operative surgical plan for a patient, the pre-operative surgical plan comprising a target surgical parameter for soft-tissue structure, receiving a pre-operative image of the patient, the image depicting the soft-tissue structure, segmenting the pre-operative image of the patient to produce a segmented pre-operative patient image, the segmented pre-operative patient image comprising at least one segmented soft-tissue structure, estimating a pre-operative passive tension for the segmented soft-tissue structure, wherein the estimated pre-operative passive tension is based at least partially on the segmented pre-operative patient image, and estimating a post-operative passive tension for the segmented soft-tissue structure, wherein the estimated post-operative passive tension is based at least partially on the estimated pre-operative passive tension and the target surgical parameter.

In some examples, software may be stored on a non-transitory computer-readable storage medium having instructions stored thereon that, when executed by one or more processors of an electronic computing device, performs a method for estimating a muscle quality of a muscle or muscle group, the method comprising: receiving an image of a patient, the image at least partially depicting a muscle or muscle group, segmenting the image of the patient to produce a segmented patient image, the segmented patient image comprising at least one segmented muscle or segmented muscle group, and estimating a muscle quality for the segmented muscle or segmented muscle group; wherein the estimated muscle quality is based at least partially on the segmented patient image.

In some examples, software may be stored on a non-transitory computer-readable storage medium having instructions stored thereon that, when executed by one or more processors of an electronic computing device, performs a method for estimating pathing of a muscle or muscle group, the method comprising: receiving an image of a patient, the image at least partially depicting at least a portion of a muscle or muscle group, segmenting the image of the patient to produce a segmented patient image, the segmented patient image comprising at least one segmented muscle or segmented muscle group, and estimating a pathing for the segmented muscle or segmented muscle group; wherein the estimated pathing is based at least partially on the segmented patient image.

While the present invention has been illustrated by the description of the examples thereof, and while the examples have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

What is claimed is:

1. A method implemented on an electronic computing device for estimating a force potential of a muscle or muscle group, the method comprising:
receiving an image of a patient, the image depicting at least a portion of a muscle or muscle group;
segmenting the image of the patient to produce a segmented patient image, the segmented patient image comprising at least one segmented muscle or segmented muscle group; and
estimating a muscle force potential for the segmented muscle or segmented muscle group; and
estimating at least one muscle characteristic for the segmented muscle or segmented muscle group,
wherein the estimated muscle force potential is based at least partially on the at least one muscle characteristic,
wherein the at least one muscle characteristic comprises:
muscle volume; and
a pennation angle, and
wherein the estimated muscle force potential is based at least partially on the segmented patient image.

2. The method of claim 1, wherein the pennation angle is estimated using a computational flow simulation.

3. The method of claim 2, wherein the pennation angle is estimated using a fast Fourier transform on a muscle region and finding a direction of the highest spatial frequency.

4. The method of claim 1, wherein the estimated muscle force potential is expressed using a Z-score.

5. A method implemented on an electronic computing device for estimating a passive tension of a soft-tissue structure, the method comprising:
receiving an image of a patient, the image depicting at least a portion of a soft tissue structure;
segmenting the image of the patient to produce a segmented patient image, the segmented patient image comprising at least one segmented soft-tissue structure;
estimating a passive tension for the segmented soft-tissue structure; and
estimating at least one soft-tissue characteristic for the segmented soft-tissue structure,
wherein the estimated passive tension is based at least partially on the at least one soft-tissue characteristic,
wherein the estimated passive tension is based at least partially on the segmented patient image.

6. The method of claim 5, wherein the soft tissue characteristic is estimated using a machine learning model or neural network.

7. The method of claim 5, wherein the soft tissue characteristic comprises one or more of a length of a muscle, an amount of connective tissue within the muscle, a fraction of connective tissue volume within the muscle volume, an image intensity of the connective tissue relative to the muscle tissue, a spatial distribution and/or density of connective tissue, a volume of a tendon associated with the muscle, and/or a cross-sectional area of a tendon associated with the muscle and wherein the soft tissue characteristic comprises one or more of a bulk volume of the structure, a shape of the structure, an average thickness of the structure, a thickness distribution of the structure, an image intensity of the structure, and/or a texture of the image.

8. The method of claim 5, wherein the estimated passive tension is expressed using a Z-score.

9. The method of claim 5, wherein the passive tension of a soft-tissue structure is a post-operative passive tension of a soft-tissue structure, the method further comprising:
receiving a pre-operative surgical plan for a patient, a pre-operative surgical plan comprising a target surgical parameter for soft-tissue structure;
segmenting a pre-operative image of the patient to produce a segmented pre-operative patient image, the segmented pre-operative patient image comprising at least one segmented soft-tissue structure;
estimating a pre-operative passive tension for the segmented soft-tissue structure, wherein the estimated pre-operative passive tension is based at least partially on the segmented pre-operative patient image;
estimating a post-operative passive tension for the segmented soft-tissue structure, wherein the estimated post-operative passive tension is based at least partially on the estimated pre-operative passive tension and the target surgical parameter; and
estimating a soft tissue characteristic, and the estimated pre-operative passive tension is based at least partially on the soft tissue characteristic.

10. The method of claim 9, wherein the soft tissue characteristic comprises one or more of a length of a muscle, an amount of connective tissue within the muscle, a fraction of connective tissue volume within the muscle volume, an image intensity of the connective tissue relative to the muscle tissue, a spatial distribution and/or density of connective tissue, a volume of a tendon associated with the muscle, and/or a cross-sectional area of a tendon associated with the muscle, wherein the soft tissue characteristic comprises one or more of a bulk volume of the structure, a shape of the structure, an average thickness of the structure, a thickness distribution of the structure, an image intensity of the structure, and/or a texture of the image.

11. The method of claim 9, wherein the target surgical parameter comprises a target joint offset, a target length adjustment, an acetabular cup position, an acetabular cup orientation, a stem size, a stem position, a stem orientation, a location of a femoral neck resection, a muscle on which to perform a muscle release, an amount of muscle release performed on a muscle, a location of a capsule to cut, an amount of a capsule to remove, a tibial tray size, a tibial tray position, a tibial tray orientation, a tibial tray spacer thickness, a femoral component size, a femoral component position, and/or a femoral component orientation.

12. The method of claim 9, wherein the post-operative passive tension is estimated using a post-operative virtual model of the patient's post-operative joint.

13. A method implemented on an electronic computing device for developing a pre-operative plan for a patient, the method comprising:
receiving a pre-operative image of the patient, the image depicting at least a portion of a soft-tissue structure;
segmenting the pre-operative image of the patient to produce a segmented pre-operative patient image, the segmented pre-operative patient image comprising at least one segmented soft-tissue structure;
estimating a pre-operative passive tension for the segmented soft-tissue structure, wherein the estimated pre-operative passive tension is based at least partially on the segmented pre-operative patient image;
estimating a soft tissue characteristic, and the estimated pre-operative passive tension is based at least partially on the soft tissue characteristic; and
determining at least one target surgical parameter based at least partially on the estimated pre-operative passive tension for the segmented soft-tissue structure.

14. The method of claim 13, wherein the soft tissue characteristic comprises one or more of a length of a muscle, an amount of connective tissue within the muscle, a fraction of connective tissue volume within the muscle volume, an image intensity of the connective tissue relative to the muscle tissue, a spatial distribution and/or density of connective tissue, a volume of a tendon associated with the muscle, and/or a cross-sectional area of a tendon associated with the muscle and wherein the soft tissue characteristic comprises one or more of a bulk volume of the structure, a shape of the structure, an average thickness of the structure, a thickness distribution of the structure, an image intensity of the structure, and/or a texture of the image.

15. The method of claim 13, wherein the target surgical parameter comprises a target joint offset, a target length adjustment, an acetabular cup position, an acetabular cup orientation, a stem size, a stem position, a stem orientation, a location of a femoral neck resection, a muscle on which to perform a muscle release, an amount of muscle release performed on a muscle, a location of a capsule to cut, an amount of a capsule to remove, a tibial tray size, a tibial tray position, a tibial tray orientation, a tibial tray spacer thickness, a femoral component size, a femoral component position, and/or a femoral component orientation.

16. The method of claim 13, wherein the target surgical parameter is such that a post-operative passive tension for the segmented soft-tissue structure is substantially the same as the pre-operative passive tension and the post-operative passive tension of the segmented soft-tissue structure is substantially the same as those on a contralateral side.

17. The method of claim 13, wherein the target surgical parameter is such that a post-operative passive tension is a relative or absolute change in passive tension defined by a user.

18. The method of claim 13, wherein the target surgical parameter is such that post-operative passive tension is substantially the same as an ideal passive tension estimated for a patient.

19. The method of claim 13, wherein the method further comprises estimating a pre-operative or post-operative active tension.

20. A method implemented on an electronic computing device for estimating pathing of a muscle or muscle group, the method comprising:
receiving an image of a patient, the image at least partially depicting at least a portion of a muscle or muscle group;
segmenting the image of the patient to produce a segmented patient image, the segmented patient image comprising at least one segmented muscle or segmented muscle group;
estimating a pathing for the segmented muscle or segmented muscle group;
estimating at least one muscle characteristic for the segmented muscle or segmented muscle group; and
estimating at least one bone characteristic,
wherein the at least one bone characteristic comprises a shape of a bone, a location of an origin point, a location of an insertion point, a shape of a bone at an origin point, a shape of a bone at an insertion point, and/or any other bony structure that the muscle wraps around, and
wherein the estimated pathing is based at least partially on the segmented patient image.
